(19) United States
(12) Patent Application Publication
(10) Pub. No.: US 2010/0196464 A1
(43) Pub. Date: Aug. 5, 2010

(54) ORLISTAT PHARMACEUTICAL FORMULATIONS

(75) Inventors: Soma Sekhar Kothamasu, Guntur (IN); Arti Sah, Jamshedpur (IN); Uma Sowjanya Asapu, Hyderabad (IN); Maheswara Reddy Arumalla, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LIMITED, Hyderabad 500 016, Andhra Pradesh (IN); DR. REDDY'S LABORATORIES, INC., Bridgewater, NJ (US)

(21) Appl. No.: 12/678,552

(22) PCT Filed: Sep. 17, 2008
(86) PCT No.: PCT/US08/76641

§ 371 Date: Mar. 17, 2010

(30) Foreign Application Priority Data
Sep. 17, 2007 (IN) 2070/CHE/2007

(51) Int. Cl. A61K 9/48; A61K 31/337; A61K 9/14

(52) U.S. Cl. 424/451; 514/449; 424/489

(57) ABSTRACT

Solid pharmaceutical formulations are prepared using compositions comprising orlistat and having average particle sizes less than about 250 μm.

The present invention relates to pharmaceutical compositions comprising orlistat, including its salts, solvates, polymorphs, racemic mixtures, enantiomers, and mixtures thereof. The invention also relates to pharmaceutical formulations comprising compositions of orlistat or its salts. Further the invention also relates to processes for preparation of the compositions and formulations of orlistat, and their methods of use.

Tetrahydrolipstatin (“THL”) is an inhibitor of pancreatic lipase and is known under the officially adopted name “orlistat.” Orlistat has a chemical name (S)-1-[[(2S,3S)-3-hexyl-4-oxo-2-oxetanyl]methyl]-dodecyl ester and is structurally represented by (1).

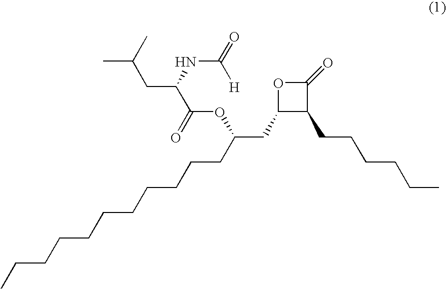

The empirical formula of the compound is C₂₉H₅₃NO₅, and its molecular weight is 495.7. It is a single diastereomeric molecule that contains four chiral centers, with a negative optical rotation in ethanol at 529 nm.

Orlistat is a white to off-white crystalline powder. Orlistat is practically insoluble in water, freely soluble in chloroform, and very soluble in methanol and ethanol. Orlistat has no pKₐ within the physiological pH range.

Orlistat mainly acts by reducing the absorption of amount of fats taken up by the patient, which leads to excretion of the unabsorbed fats in the feaces. Reduction in the absorption of the fats leads to reduction in body weight.

Orlistat is available in hard gelatin capsules in the two strengths of 120 mg and 60 mg, respectively marketed as XENICAL™ and ALLI™.

XENICAL and ALLI products are used in the treatment of obesity management, including weight loss and weight maintenance, when used in conjunction with a reduced-calorie diet. The products are also indicated to reduce the risk for weight regain after prior weight loss.

Orlistat is disclosed in U.S. Pat. No. 4,598,089. U.S. Pat. Nos. 6,004,996 and 6,730,319, U.S. Patent Application Publication No. 2008/0021092, International Application Publication Nos. WO 1993/4787, WO 2000/09122, WO 2000/09123, WO 2000/013667 WO 2002/00201, WO 2003/090742, WO 2006/035296, WO 2006/11080, WO 2007/021073, WO 2008/034533, WO 2008/000420, and WO 2007/123338, and Russian Patent Nos. 2271808, 2239428, 2248218, and 2241462, disclose various pharmaceutical formulations of orlistat.

Orlistat is a BCS (Biopharmaceutical Classification System) class II compound and is insoluble in water. Orlistat is a waxy, fluffy and sticky material having a low melting point about 44° C. Due to its nature and low melting point, orlistat is prone to physical instability, and also processing of orlistat into pharmaceutical formulations is difficult.

To address the instability issues of orlistat, various methods have been adopted. These methods have been observed to be tedious and costly. Hence there is still a need for simple formulations, and simple processes to prepare the formulations, which are cost-effective.

### SUMMARY

The present invention relates to pharmaceutical compositions comprising orlistat, including its salts, solvates, polymorphs, racemic mixtures, enantiomers, and mixtures thereof. The invention also relates to solid pharmaceutical formulations comprising compositions of orlistat or its salts. Further the invention relates to processes for preparation of the compositions and formulations of orlistat.

In an embodiment the invention includes pharmaceutical compositions comprising orlistat having average particle sizes less than 250 μm, or less than about 200 μm, or less than about 150 μm.

In embodiments, pharmaceutical compositions comprising orlistat and at least one pharmaceutically acceptable excipient, wherein average particle sizes of compositions is less than about 250 μm, or less than about 200 μm, or less than about 150 μm

In another embodiment the invention includes pharmaceutical compositions comprising orlistat and at least one pharmaceutically acceptable excipient, wherein average particle sizes of the compositions are more than about 2 mm.

In an embodiment, pharmaceutical formulations comprising compositions of orlistat, wherein the average sizes of the compositions is less than about 250 μm, or less than about 200 μm, or less than about 150 μm.

In an embodiment, pharmaceutical formulations comprising compositions of orlistat, wherein the average sizes of the compositions are more than about 2 mm, or more than about 2.5 mm, or more than about 3 mm.

In an embodiment, the invention relates to stable pharmaceutical compositions comprising orlistat.

In an embodiment the invention includes pharmaceutical formulations comprising orlistat, wherein the concentration of delactone orlistat impurity is less than about 2%, or less than about 1.5%, by weight of a label orlistat content.

Further embodiments of the invention include pharmaceutical formulations comprising orlistat, wherein the concentration of total impurities is less than about 4%, or less than about 2%, by weight of a label orlistat content.

In another embodiment the invention relates to stable pharmaceutical formulations comprising compositions of orlistat.

An embodiment of the present invention includes bulk densities and tapped densities of orlistat or its salts which are in the range of about 0.2 g/ml to about 0.6 g/ml, and about 0.3 g/ml to about 0.8 g/ml, respectively.

In an embodiment the present invention includes bulk densities and tapped densities of final blends comprising orlistat and at least one pharmaceutical excipient in the range of about 0.2 g/ml to about 0.6 g/ml, and about 0.3 g/ml to about 0.8 g/ml, respectively.

In an embodiment the present invention further includes methods of using the pharmaceutical formulations in the treatment of obesity and related disorders.

### BRIEF DESCRIPTION OF THE DRAWING

FIG. 1 shows comparative powder X-ray diffraction (XRD) patterns for the formulation prepared according to Example 6, wherein A represents orlistat, P represents a placebo formulation, B represents the formulation as initially prepared, and C represents the formulation after storage at 30° C. and 75% relative humidity for 1 month.

### DETAILED DESCRIPTION

The present invention relates to pharmaceutical formulations comprising orlistat, including its pharmaceutically acceptable salts, solvates, polymorphs, racemic mixtures, enantiomers, and mixtures thereof.

The numbers of people suffering from obesity have tremendously increased in recent years. The prevalence of obesity increased not only in adults but also, to a marked extent, in adolescents. Increases in obesity lead to various disorders such as hypertension, cardiac failure and diabetes mellitus.

Orlistat is a reversible lipase inhibitor. It exerts its therapeutic activity in the lumen of the stomach and small intestine by forming covalent bonds with active serine sites of gastric and pancreatic lipases. The inactivated enzymes are thus unavailable to hydrolyze dietary fat in the form of triglycerides into absorbable free fatty acids and monoglycerides; undigested fats are not absorbed, and the resulting caloric deficit may have a positive effect on weight control.

The term “average particle size” refers to particle sizes represented by D(0.5), wherein 50% of particles in a powder have sizes greater than, and 50% of particles have sizes less than, a specified value.

The term “composition” refers to a mixture comprising orlistat and at least one pharmaceutically acceptable excipient, wherein the compositions can be in the form of powders, granules, pellets, particles, or minitablets.

The term “formulation” refers to a pharmaceutical dosage form containing a composition comprising orlistat or a salt thereof. The pharmaceutical formulations of the present invention can be prepared as solid oral dosage forms. Solid oral dosage forms include, for example, tablets, caplets, capsules (hard or soft gelatin capsules), orally disintegrating dosage forms, chewable dosage forms, pills, granules, sachets, and the like.

The term “stability” includes both physical and chemical stability, suitable for commercial activities. This implies maintenance of original formulation specifications after manufacture, for a period of at least about six months, or at least about 1 year, or at least about 2 years, to the extent necessary for sale and use of the formulation.

“Physical stability” is evidenced by maintenance of initial formulation XRD patterns during storage and use.

Pharmaceutically acceptable salts of orlistat include salts prepared from pharmaceutically acceptable non-toxic bases including inorganic bases and organic bases, or acids including inorganic and organic acids. Salts derived from inorganic bases include aluminum, ammonium, calcium, copper, ferric, ferrous, lithium, magnesium, manganic, manganous, potassium, sodium, and zinc salts, and the like. Salts derived from organic bases include salts of primary, secondary, and tertiary amines, and substituted amines including naturally occurring substituted amines.

“Pharmaceutically acceptable acid addition salt” refers to a salt formed with a drug compound and inorganic acids such as hydrochloric acid, sulfuric acid, phosphoric acid and the like, and with organic acids such as acetic acid, propionic acid, glycolic acid, pyruvic acid, oxalic acid, maleic acid, malonic acid, succinic acid, filmaric acid, tartaric acid, citric acid, benzoic acid, cinnamic acid, mandelic acid, methanesulfonic acid, ethanesulfonic acid, p-toluenesulfonic acid, salicyclic acid and the like. These lists are not all-inclusive, merely giving a few examples.

Various parameters impacting the preparation of solid oral dosage forms include the physical properties of active ingredients as well as those of the final blends of active ingredients with excipients, wherein the physical properties include flow properties, particle sizes (such as determined by sieve analyzers, electrical conductance instruments such as a Coulter counter, and laser light diffraction particle size analyzers such as the instruments available from Malvern Instruments, Ltd., Malvern, Worcestershire, United Kingdom), bulk densities and tapped densities, compressibility indexes, Hausner ratios (determined by USP density apparatus), flow properties (such as determined by Flowdex apparatus), etc.

Particle sizes play an important role in establishing solubility. As the particle sizes are reduced, the surface areas of the individual particles of orlistat increase, thus a greater amount of orlistat can be solubilized for obtaining a therapeutic effect, which leads to higher therapeutic efficacy.

The percent of particles with different dimensions that exist in a powder is called the particle size distribution. It is represented in certain ways. Particle size is the maximum dimension of a particle, normally expressed in units of μm. Particle size distributions can be expressed in terms of D(0.1), D(0.5), D(0.9) and D [4,3]. The D(0.1), D(0.5) and D(0.9) represent the 10th, median or the 50th percentile, and the 90th percentile of the particle size distribution, respectively. They can be expressed as volume, weight, or surface percentages. For example when measured by volume, D(0.1), D(0.5), D(0.9) is a value of the distribution such that 10%, 50%, 90% by volume of the particles have a size of this value or less, or is the percentage of particles smaller than that size. D(0.5) is also known as median diameter of particle. It is one of the important parameters representing characteristics of particle of powder. For a sample, if D(0.5)=5 μm, it means that 50% of the particles are smaller than 5 μm. Similarly, if D(0.1)=5 μm, 10% by volume of the particles are less than or equal to 5 μm, and if D(0.9)=5 μm, 90% of the particles are less than or equal to 5 μm. D[4,3] is the volume moment mean of the particles or the volume weighted particle size.

In an embodiment the invention includes pharmaceutical compositions comprising orlistat having average particle sizes less than 250 μm, or less than about 200 μm, or less than about 150 μm, or less than about 100 μm.

In embodiments, pharmaceutical compositions comprise orlistat and at least one pharmaceutically acceptable excipient, wherein average particle sizes of compositions are less than about 250 μm, or less than about 200 μm, or less than about 150 μm, or less than about 100 μm.

In another embodiment the invention includes pharmaceutical compositions comprising orlistat and at least one pharmaceutically acceptable excipient, wherein average particle sizes of the compositions are more than about 2 mm, or more than about 2.5 mm, or more than about 3 mm. Sizes up to about 6 mm will generally be useful.

In an embodiment, pharmaceutical formulations comprise compositions of orlistat, wherein the average sizes of the compositions are less than about 250 μm, or less than about 200 μm, or less than about 150 μm, or less than about 100 μm.

In an embodiment, pharmaceutical formulations comprise compositions of orlistat, wherein the average sizes of the composition particles are more than about 2 mm, or more than about 2.5 mm, or more than about 3 mm. The particles generally will not be larger than about 6 mm.

Other important physicochemical characteristics of powders are the density properties such as bulk and tapped density, weight variation and flow properties such as angle of repose. Bulk density is the undisturbed packing density of that substance and tapped bulk density relates to the packing density after tapping a bed of substance until no change in the packing density is seen. Bulk density and tapped density, can be determined using compendial bulk density apparatus, such as the method given in Test 616 “Bulk Density and Tapped Density,” United States Pharmacopeia 29, United States Pharmacopeial Convention, Inc., Rockville, Md., 2005 (“USP”). Weight variation can be determined using the method given in United States Pharmacopeia 29.

In an embodiment, the present invention provides untapped bulk densities and tapped densities of orlistat or a salt, etc. thereof in the range of about 0.2 g/ml to about 0.6 g/ml, and about 0.3 g/ml to about 0.8 g/ml, respectively.

In an embodiment of the present invention, untapped bulk densities and tapped densities of final blends comprising orlistat or a salt, etc. thereof and at least one pharmaceutical excipient are in the range of about 0.2 g/ml to about 0.6 g/ml, and about 0.3 g/ml to about 0.8 g/ml, respectively

The flowability of a composition is influenced by the particle size distribution, electrostatic properties, particle shapes and hygroscopicity. Addition of flow aids may smooth the surfaces of the particles leading to improved flowability. Moisture binds particles and hence decreases the flowability. The determination of flowability is based upon ability of the powder to fall freely through a hole in a disc. The smaller the hole through which the powder falls freely, the better is the flowability. Flowdex apparatus can be used to measure flowability and the angle of repose. The Flowdex apparatus consists of a cylinder with interchangeable discs having holes of various diameters at the bottom. The cylinder is filled with powder without packing and the diameters of the holes are successively reduced until the powder will not flow through. The Flowdex flowability rating can then be calculated by dividing 1000 by the smallest hole diameter (in mm) through which the powder will flow.

For angle of repose, after carefully overfilling the cylinder with powder and measuring the height of the powder cone (h) and the radius of the cylinder (r), the angle of repose (tan α) can be calculated as:

tan α=h÷r.

The smaller the angle of repose, the better the flowability.

| Angle of Repose | Flowability |
|---|---|
| ≦31 | Excellent |
| 32-45 | Good |
| 46-56 | Medium |
| ≧57 | Poor |

In an embodiment the invention includes pharmaceutical compositions comprising orlistat or its salts wherein angle of repose is less than about 40, or less than about 30.

In yet another embodiment the invention includes solid pharmaceutical formulations comprising orlistat or its salts wherein weight variation of units of the formulation is within the limits of ±7.5%, or ±10%, of an average weight of 20 units of the formulation.

Preparation of Formulations is Influenced by Physicochemical Properties of active ingredients and other important additives. Due to the low melting point of orlistat at about 44° C., conventional dosage forms, for example tablets and capsules, cannot be easily formulated from powder mixtures due to picking and sticking problems during tablet compression or encapsulation. Further, due to its nature, orlistat undergoes both hydrolytic and thermal degradation. The processes of preparing formulations may involve several operations such as milling, sieving, wet or dry granulation, slugging, encapsulation, etc. Mechanical energy produced in such operations can be imparted to materials being processed. Often, this leads to melting, deformulation or inactivation of the drug substance.

Due to the instability of orlistat, impurities may be generated during processing to prepare pharmaceutical formulations or during stability testing. Some of the impurities that have been identified for orlistat are described below.

Orlistat can undergo hydrolytic degradation to form compounds including:

1) 5-(2-Formylamino-4-methyl-pentanoyloxy)-2-hexyl-3-hydroxyhexadecanoic acid, having structural formula (2).

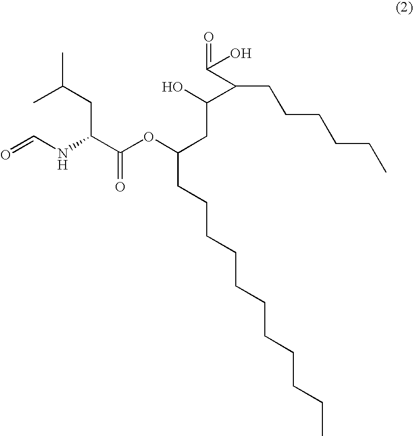

2) 2-Formylamino-4-methyl-pentanoic acid, having structural formula (3).

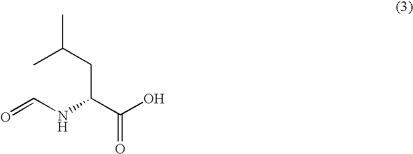

3) 3-Hexyl-4-(2-hydroxytridecyl)oxetan-2-one having structural formula (4).

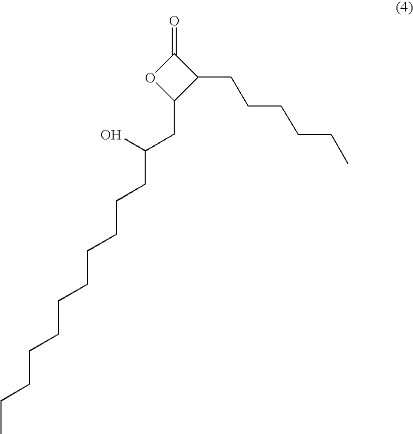

4) 2-Hexyl-3,5-dihydroxyhexadecanoic acid, having structural formula (5).

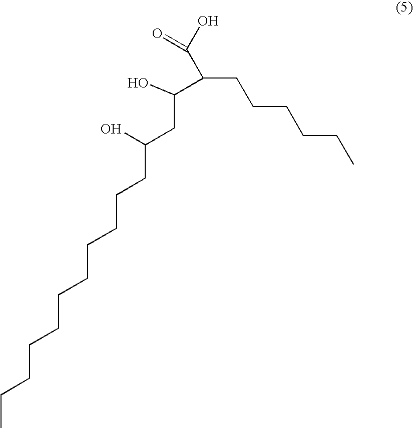

Other potential impurities which may be formed as degradation products in the formulations are described below:

5) Delactone orlistat, having structural formula (6).

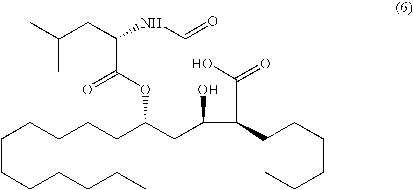

6) Deformyl N-pivaloyl orlistat having structural formula (7).

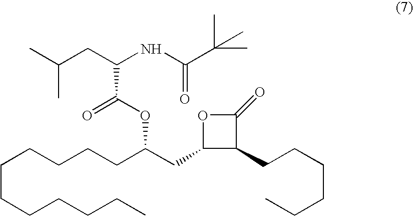

7) A dimeric impurity having structural formula (8).

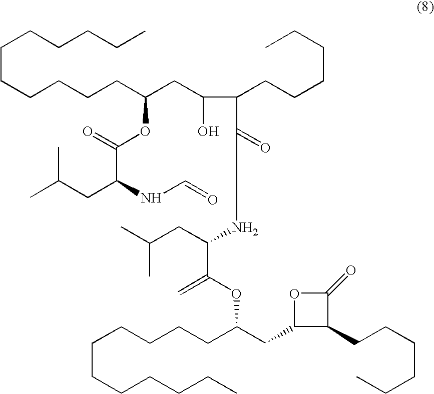

8) CBz-orlistat having structural formula (9).

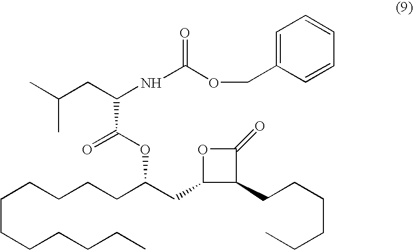

9) Orlistat isomers, including orlistat (S,R,R,R) isomer (10), orlistat (S,R,S,S) (11), and Orlistat (S,S,R,R) isomer (12).

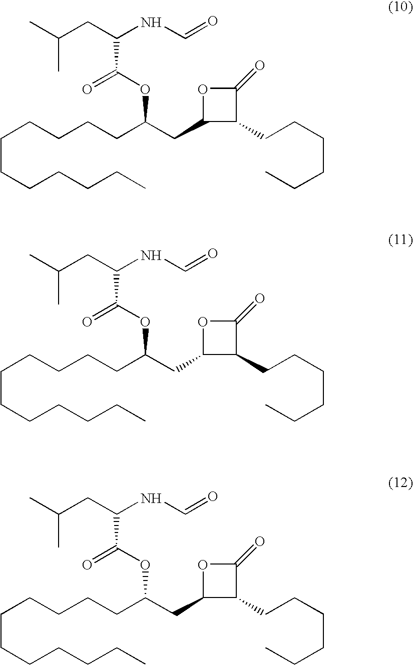

10) N,N-dicyclohexyl urea (DCU) having structural formula (13).

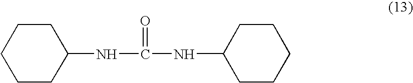

11) Unidentified impurities having RRT values of 0.59, 1.12 and 1.24, in the HPLC procedure described below.

In an embodiment the invention includes stable pharmaceutical formulations comprising orlistat, including its salts, etc.

In an embodiment the invention includes stable pharmaceutical formulations comprising orlistat or its salts, etc., wherein moisture content of the formulation is less than about 7% w/w.

In yet another embodiment the invention includes stable formulations comprising orlistat, wherein formulations retain their initial XRD patterns during storage under commercially relevant conditions for a commercially relevant time.

In an embodiment the invention includes stable pharmaceutical formulations comprising orlistat, wherein the concentration of delactone orlistat impurity is less than about 2%, or less than about 1.5%, by weight of a label orlistat content.

Further embodiments of the invention includes stable formulations comprising orlistat, wherein total impurities are less than about 4%, or less than about 2%, by weight of an initial orlistat content.

In an embodiment the invention relates to analytical methods for analysis of impurities using high performance liquid chromatography (HPLC), wherein a method comprises the following:

Buffer solution: 1 g of sodium perchlorate monohydrate was dissolved in 1 L of water and pH was adjusted to 2.5 with dilute orthophosphoric acid solution. The solution was filtered through a 0.45 μm filter.

Mobile phase A: Buffer (pH 2.5) and methanol (800:200 by volume).

Mobile phase B: Acetonitrile.

Diluent: Acetonitrile.

Chromatographic system:

a) The liquid chromatograph is equipped with a 210 nm UV detector.

b) Zorbax Eclipse XDB-C8, 150×4.6 mm, 5 μm column.

c) Temperature: 30° C.

d) Flow rate: 1.5 mL per minute.

e) Injection volume: 10 μL.

f) Run time: 65 minutes.

The relative retention times (RRT) of various impurities are tabulated below.

| Impurity | RRT* |
|---|---|
| N,N-dicyclohexyl urea (DCU) | 0.08 |
| Delactone orlistat | 0.59 |
| Orlistat isomer (SRRR) | 1.07 |
| Orlistat isomer (SRSS) | 1.1 |
| Unidentified impurity at RRT 1.12 | 1.12 |
| Unidentified impurity at RRT 1.24 | 1.24 |
| Deformyl N-pivaloyl orlistat | 1.34 |
| CBz-orlistat | 1.39 |
| Dimeric impurity | 2.27 |

*Relative retention time, where orlistat = 1.

In an embodiment the present invention provides pharmaceutical formulations comprising orlistat or its salts, wherein the formulations are in solid oral dosage forms, such as tablets, minitablets, capsules, lozenges, pills, and granules.

The solid dosage forms may include any number of excipients, including, but not limited to, diluents or fillers, binding agents, disintegrants, coloring agents, lubricating agents, glidants, solvents, film-forming agents and wetting agents.

### Diluents/Fillers:

Various useful fillers or diluents include but are not limited to starches, lactose, mannitol (Pearlitol™ SD200), cellulose derivatives, confectioner's sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH101, PH102, PH301, PH302 and PH-F20, PH-112 microcrystalline cellulose PH114, and microcrystalline cellulose PH112. Other useful diluents include but are not limited to croscarmellose, sugar alcohols such as mannitol, sorbitol and xylitol, calcium carbonate, magnesium carbonate, dibasic calcium phosphate, and tribasic calcium phosphate.

### Binders:

Various useful binders include but are not limited to hydroxypropylcelluloses (Klucel™-LF), hydroxypropylcelluloses (Klucel EXF) hydroxypropyl methylcelluloses or hypromelloses (Methocel™), polyvinyl pyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

### Disinteqrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™, FMC-Asahi Chemical Industry Co., Ltd.), crospovidones, examples of commercially available crospovidone products including but not limited to crosslinked povidones, Kollidon™ CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropylcelluloses include but are not limited to low-substituted hydroxy propylcellulose LH11, LH21, LH31, LH22, LH32, LH20, LH30, LH32 and LH33 (all manufactured by Shin-Etsu Chemical Co., Ltd.). Other useful disintegrants include sodium starch glycolate Type A, colloidal silicon dioxide 200, and starches.

### Coloring Agents:

Coloring agents can be used to color code the formulation, for example, to indicate the type and dosage of the therapeutic agent therein. Suitable coloring agents include, without limitation, natural and/or artificial materials such as FD&C coloring agents, natural juice concentrates, pigments such as titanium oxide, silicon dioxide, iron oxides, and zinc oxide, combinations thereof, and the like.

### Lubricants:

An effective amount of any generally accepted pharmaceutical tableting lubricant can be added to assist in compressing tablets. Useful lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and combinations thereof.

### Glidants:

One or more glidant materials, which improve the flow of a powder blend and minimize the dosage form weight variation, can be used. Useful glidants include but are not limited to silicone dioxide, talc, and combinations thereof.

### Solvents:

Solvents that can be used in processing include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, and mixtures thereof.

The final formulation may be coated or uncoated. For coating, additional excipients such as film-forming polymers, plasticizers, antiadherents and opacifiers are frequently used.

### Film-Forming Agents:

Various useful film-forming agents include but are not limited to cellulose derivatives such as soluble alkyl- or hydroalkylcellulose derivatives including methyl celluloses, hydroxymethyl celluloses, hydroxyethyl celluloses, hydroxy propyl celluloses, hydroxymethylethyl celluloses, hydroxypropyl methyl celluloses, sodium carboxymethyl celluloses, etc., acidic cellulose derivatives such as cellulose acetate phthalates, cellulose acetate trimellitates and methylhydroxy propylcellulose phthalates, polyvinyl acetate phthalates, etc., insoluble cellulose derivatives such as ethyl celluloses and the like, dextrins, starches and starch derivatives, polymers based on carbohydrates and derivatives thereof, natural gums such as gum Arabic, xanthans, alginates, polyacrylic acid, polyvinyl alcohol, polyvinyl acetate, polyvinylpyrrolidones, polymethacrylates and derivatives thereof (Eudragit™), chitosan and derivatives thereof, shellac and derivatives thereof, waxes and fat substances.

### Wetting Agents:

Wetting agents that are useful include anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium dodecyl sulfate (SLS or SDS), cationic surfactants such as cetylpyridinium chloride monohydrate and hexadecyl trimethylammonium bromide, nonionic surfactants such as N-decanoyl-N-methylglucamine, octyl a-D-glucopyranoside, n-Dodecyl b-D-maltoside (DDM), polyoxyethylene sorbitan esters like polysorbates and the like, plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like.

Solvents that may be used in layering or coating include but are not limited to: aqueous solvents such as water; organic volatile solvents such as acetaldehyde, acetone, benzene, carbon disulphide, carbon tetrachloride, 1,2 dichloroethane, dichloromethane, N,N-dimethylformamide, 1,4-dioxane, epichlorhydrin, ethyl acetate, ethanol, ethyl ether, ethylene glycol, 2-ethoxyethanol (acetate), formaldehyde, isopropanol, methanol, methyl n-butyl ketone, methyl ethyl ketone, 2-methoxyethanol (acetate), perchloroethylene, toluene, 1,1,1-trichloroethane, trichloroethylene; and the like, and mixtures thereof.

If desired, the films may contain additional adjutants for coating processing such as plasticizers, polishing agents, colorants, pigments, antifoam agents, opacifiers, antisticking agents, and the like.

In embodiments of the present invention, equipment suitable for processing the pharmaceutical formulations include one or more of mechanical sifters, granulators, blenders, roller compactors, compression machines, rotating bowls or coating pans, fluid bed processors, etc.

In embodiments of the present invention, the pharmaceutical formulations may be processed by direct compression, dry granulation or wet granulation, extrusion followed by spheronization, etc.

In an aspect the present invention provides processes for preparing pharmaceutical formulations comprising orlistat, its salts, etc., wherein an embodiment comprises:

a) sifting active ingredient;

b) sifting excipients;

c) dry mixing active and excipients in a granulator;

d) optionally, compacting the step c) mixture and subsequently milling the compacts and sifting to form granules;

e) optionally, dissolving or dispersing a binder in a suitable solvent and granulating the step c) mixture;

f) drying granules;

g) sifting granules and extragranular excipients;

h) blending sized granules, or dry mixed excipients from step c), with sifted extragranular excipients (except lubricants);

i) adding sifted lubricant to step h) and blending; and

j) filling a lubricated blend from i) into capsules, or alternatively compressing into tablets and optionally filling tablets into capsules.

Dosage forms prepared by a process can be subjected to in vitro dissolution testing, such as according to Test 711 “Dissolution” in United States Pharmacopeia 29, United States Pharmacopeial Convention, Inc., Rockville, Md., 2005 to determine the extent and rate at which the active substance is released from the dosage forms, and the content of the active substance can be determined in solutions using techniques such as high performance liquid chromatography.

In an embodiment the present invention includes use of packaging materials such as containers and lids of high-density polyethylene (HYPE), low-density polyethylene (LDPE) and or polypropylene or polyethylene and/or glass, and blisters or strips composed of aluminum or high-density polypropylene, polyvinyl chloride, polyvinylidene dichloride, or aluminum/aluminum foil blisters or polyvinyl chloride/polyethylene/polyvinylidene dichloride (PVC/PE/PVDC) film packaging.

Certain specific aspects and embodiments of this invention are described in further detail by the examples below, which examples are provided only for purposes of illustration and are not intended to limit the scope of the invention in any manner.

### Example 1

### Pharmaceutical Formulations Comprising Orlisat 120 Mg

| Ingredient | mg/Capsule |
|---|---|
| Orlistat ‡ | 120 |
| Microcrystalline cellulose PH112 | 83.3 |
| Sodium starch glycolate (Type A) | 5.42 |
| Sodium lauryl sulphate (SLS) | 2.08 |
| Povidone (PVP-30) | 4.17 |
| Colloidal silicon dioxide (200) | 2.5 |
| Purified talc | 2.5 |

‡ Particle size distribution of orlistat used in the formulation:

| Parameter | μm |
|---|---|
| D(0.1) | 1.8 |
| D(0.5) | 8.6 |
| D(0.9) | 24.5 |

Manufacturing process:
a) Orlistat, microcrystalline cellulose and sodium starch glycolate were sifted through an ASTM #30 mesh sieve.

b) Sifted ingredients were blended uniformly in an octagonal blender.

c) Talc and colloidal silicon dioxide were sifted through an ASTM #40 mesh sieve.

d) Step b) and step c) materials were blended uniformly in an octagonal blender.

e) Blend from step d) was filled into size ‘0’ hard gelatin capsules.

f) Capsules were packaged in PVC/PVDC 90 gsm (polyvinyl chloride having a 90 gram per square meter coating of polyvinylidene dichloride) blister pack.

Particle size distribution for the final blend has been determined and the data are given below:

| Parameter | Result |
|---|---|
| D(0.1) | 9.55 μm |
| D(0.5) | 59.63 μm |
| D(0.9) | 204.86 μm |

### Example 2

### Pharmaceutical Formulations Comprising Orlisat 120 mg

| Ingredient | mg/Tablet |
|---|---|
| Orlistat | 120 |
| Microcrystalline cellulose PH101 | 93.6 |
| Sodium starch glycolate Type A | 7.2 |
| Povidone (K-30) | 12 |
| Water* | 0.15 |
| Sodium lauryl sulphate | 7.2 |
| Purified talc | 0.24 |

*Evaporates during processing.

Manufacturing Process:

a) Orlistat was sifted through an ASTM #30 mesh sieve.

b) Microcrystalline cellulose, sodium starch glycolate, povidone and talc were sifted through an ASTM #40 mesh sieve.

c) All of the excipients, except for talc, were dry mixed in a rapid mixer granulator.

d) Sodium lauryl sulphate was dissolved in water.

e) The dry mixture from step c) was granulated using solution from step d).

f) Granules were dried at room temperature until loss on drying was less than 1.5% w/w.

g) Dry granules were sifted through an ASTM #20 mesh sieve.

h) Sized granules were lubricated by mixing with talc in a double cone blender.

i) Lubricated blend was filled into size ‘0’ hard gelatin capsules with an average fill weight of 240 mg.

### Example 3

### Pharmaceutical Formulations Comprising Orlisat 120 mg

| Ingredient | mg/Tablet |
|---|---|
| Orlistat | 120 |
| Microcrystalline cellulose PH 101 | 93.6 |
| Sodium starch glycolate Type A | 7.2 |
| Water* | 0.15 |
| Sodium lauryl sulphate | 7.2 |
| Purified talc | 0.24 |

*Evaporates during processing.

Manufacturing process was similar to that of Example 2.

### Example 4

### Pharmaceutical Formulations Comprising Orlisat 120 mg

| Ingredient | mg/Tablet |
|---|---|
| Orlistat | 40 |
| Microcrystalline cellulose PH 112 | 33.33 |
| Sodium starch glycolate Type A | 2.17 |
| Povidone | 1.67 |
| Sodium lauryl sulphate | 0.83 |
| Purified talc | 1 |
| Colloidal silicon dioxide | 1 |

Manufacturing Process:

a) Orlistat, microcrystalline cellulose, sodium starch glycolate and povidone were sifted through an ASTM #30 mesh sieve.

b) Sieved ingredients were blended uniformly in a double cone blender.

c) Colloidal silicon dioxide and talc were sifted through an ASTM #40 mesh sieve.

d) Step b) mixture was lubricated by blending with step c) materials in a double cone blender.

e) The blend of d) was compressed into mini-tablets weighing 80 mg, using 5 mm standard concave plain punches.

f) Three of the tablets were filled into a size ‘0’ hard gelatin capsule.

Particle size distribution for the final blend was determined and the data are given below:

| Parameter | Result |
|---|---|
| D(0.1) | 9.55 μm |
| D(0.5) | 59.63 μm |
| D(0.9) | 204.86 |

### Example 5

### Drug Dissolution Characteristics of Products from Examples 1-3

Dissolution Conditions:

Medium: pH 6.0 buffer with 3% of SLS and sodium chloride.

Speed: 75 rpm.

Volume: 900 mL.

Apparatus: USP Type II (paddle) apparatus.

Results are shown below, with the commercially available XENICAL® product also tested for comparison.

| | |
|---|---|
| Time | Cumulative % of Drug Dissolved |

| (minutes) | XENICAL | Example 1 | Example 2 | Example 3 |
|---|---|---|---|---|
| 15 | 56 | 53 | 27 | 54 |
| 30 | 83 | 74 | 54 | 68 |
| 45 | 94 | 84 | 78 | 87 |
| 60 | 98 | 98 | 93 | 97 |

### Example 6

### Pharmaceutical Formulations Comprising Orlisat 120 Mg

| Ingredient | mg/Capsule |
|---|---|
| Orlistat* | 120 |
| Microcrystalline cellulose PH 112 | 68.47 |
| Sodium starch glycolate Type A | 5.96 |
| Povidone | 4.58 |
| Sodium lauryl sulphate | 2.29 |
| Purified talc | 11 |
| Colloidal silicon dioxide | 1.1 |
| Magnesium stearate | 6.6 |

Particle size distribution of orlistat used in the formulation:

| Parameter | μm |
|---|---|
| D(0.1) | 18.7 |
| D(0.5) | 84.2 |
| D(0.9) | 171.8 |
| Bulk density (g/ml) | 0.424 |
| Tapped density (g/ml) | 0.571 |

Manufacturing Process:

1) Orlistat and remaining ingredients, except purified talc and magnesium stearate, were sifted through an ASTM #40 mesh sieve.

2) Step 1) materials were blended for about 10 minutes.

3) Purified talc and magnesium stearate were separately sifted through an ASTM #60 mesh sieve.

4) Step 2) materials were blended with step 3) materials for about 5 minutes.

5) The blend was filled into size ‘1’ hard gelatin capsules using a capsule filling machine.

Physical characteristics for the final blend, including particle size distribution parameters D(0.1), D(0.5), D(0.9), and D[4,3], bulk density, tap density, angle of repose, and moisture content, are given below:

| Parameter | Result |
|---|---|
| D(0.1) | 13.2 μm |
| D(0.5) | 78.5 μm |
| D(0.9) | 191.4 μm |
| D[4.3] | 91.44 μm |
| Bulk density (g/ml) | 0.434 |
| Tapped density (g/ml) | 0.594 |
| Angle of repose | 26 |
| Moisture content (%) | 3.66 |

The capsules were tested for their dissolution characteristics in 900 ml of pH 6 buffer (with 0.5% NaCl and 3% sodium lauryl sulfate) at 75 rpm stirring in USP type II apparatus, and compared with commercial XENICAL® product as a reference. The comparative data are given below:

| | |
|---|---|
| | Cumulative % of |
| Time | Drug Dissolved |

| (minutes) | Example 6 | XENICAL |
|---|---|---|
| 10 | 47 | 43 |
| 20 | 70 | 68 |
| 30 | 84 | 81 |
| 45 | 92 | 91 |
| 60 | 96 | 98 |

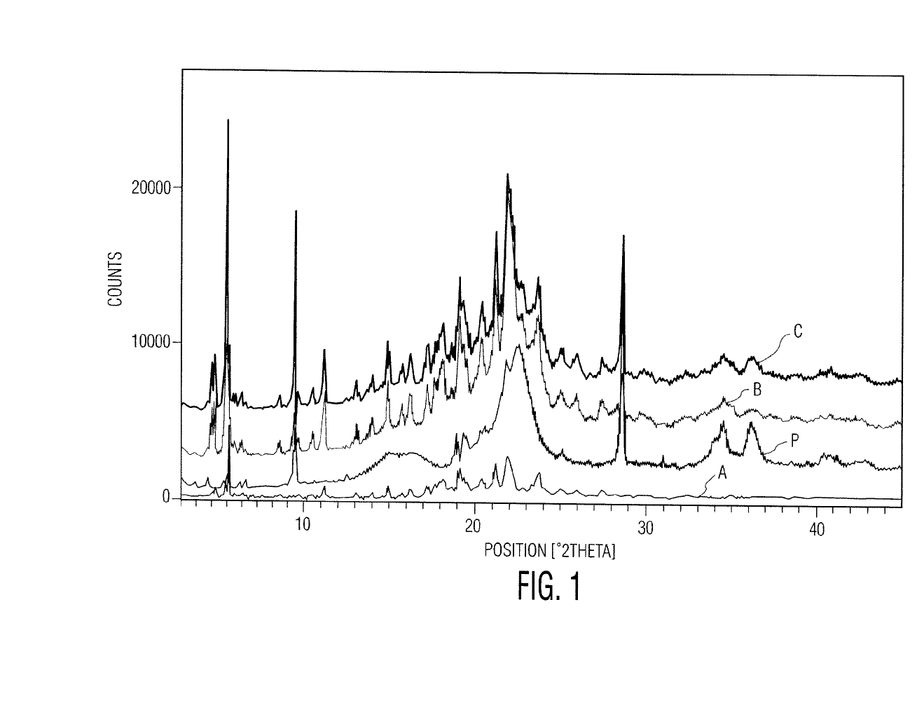

The capsules were packed in blister packages made of a polyvinyl chloride/polyethylene/polyvinylidene dichloride laminated film and aluminum foil as a backing, and blisters were stored at 30° C. and 75% relative humidity (RH) for 1 month. FIG. 1 is a comparison of powder X-ray diffraction (XRD) patterns, using copper Kα-1 radiation, of the orlistat ingredient (A), the capsule contents as originally prepared (B), and the capsule contents after storage (C). A “placebo” formulation was similarly prepared using the above ingredients, but omitting the orlistat, and the XRD pattern of the capsule contents is also shown (P). The XRD pattern of the formulation before storage matches that of the stored formulation, showing polymorphic stability.

The contents of packaged capsules stored at 30° C. and 75% RH, and 25° C. and 60% RH, for 2 months were tested for impurities by HPLC and for moisture content. Commercially available XENICAL capsules were stored at 30° C. and 75% RH and similarly analyzed. The data are given below:

| | XENICAL | | Example 6 | | | | |
|---|---|---|---|---|---|---|---|
| | | 30° C./75% RH | | 30° C./75% RH | | 25° C./60% RH | |
| Parameter | Initial | 1 Month | Initial | 1 Month | 2 Months | 1 Month | 2 Months |
| Delactone | 0.61 | 0.74 | 0.07 | 0.11 | 0.17 | 0.1 | 0.13 |
| orlistat | | | | | | | |
| Unidentified | ND* | ND* | 0.09 | 0.09 | 0.13 | 0.09 | 0.13 |
| impurity at RRT | | | | | | | |
| 1.12 | | | | | | | |
| Unidentified | ND* | ND* | 0.03 | 0.03 | 0.03 | 0.02 | 0.03 |
| impurity at RRT | | | | | | | |
| 1.24 | | | | | | | |
| Highest | 0.16 | 0.21 | ND* | 0.02 | 0.03 | 0.02 | 0.02 |
| unidentified | | | | | | | |
| impurity | | | | | | | |
| Total impurities | 1.06 | 1.34 | 0.2 | 0.26 | 0.43 | 0.24 | 0.35 |
| Moisture | 4.59 | 5.38 | 3.66 | 3.32 | — | — | — |
| content | | | | | | | |

*ND = Not detected.

### Examples 7-9

### Pharmaceutical Formulations Comprising Orlistat 120 Mg

| | Example 7 | Example 8 | Example 9 |
|---|---|---|---|
| Ingredient | mg/Capsule | | |
| Orlistat | 120 | 120 | 120 |
| Microcrystalline | 81.67 | 77.27 | 75.07 |
| cellulose PH 112 | | | |
| Sodium starch | 5.96 | 5.96 | 5.96 |
| glycolate Type A | | | |
| Povidone K-30 | 4.58 | 4.58 | 4.58 |
| Sodium lauryl | 2.29 | 2.29 | 2.29 |
| sulphate | | | |
| Purified talc | 2.75 | 2.75 | 2.75 |
| Colloidal silicon | 2.75 | 2.75 | 2.75 |
| dioxide | | | |
| Magnesium | — | 4.4 | 6.6 |
| stearate | | | |

Manufacturing process: Similar to that of example 6.

The capsules prepared were tested for variability of the fill weight. During the process of testing weight variation, an intact capsule was weighed, the contents were removed, and the empty capsule shell was weighed. The difference in the two weights is the fill weight. The procedure was repeated for an additional 19 capsules. The average, minimum and maximum of these twenty values were taken into consideration. The minimum (−) and maximum (+) deviations (from the average weight) percentages were calculated by:

Minimum deviation=((Average−Minimum)/Average)×100

Maximum deviation=((Maximum−Average)/Average)×100

| Parameter | Example 6 | Example 7 | Example 8 | Example 9 |
|---|---|---|---|---|
| Minimum deviation (%) | 5.47 | 10.55 | 10.25 | 12.47 |
| Maximum deviation (%) | 3.22 | 18.18 | 12.37 | 7.13 |

Example 6 has been observed to be well within the limits ±10%.

### Example 10

### Pharmaceutical Formulation Comprising Orlistat 120 Mg

| Ingredient | mg/Capsule |
|---|---|
| Orlistat | 120 |
| Dibasic calcium phosphate anhydrous | 103.5 |
| Alginic acid | 12.5 |
| Sodium alginate | 5 |
| Polysorbate 80 (Tween ™ 80) | 6 |
| Colloidal silicon dioxide | 3 |
| Water | 150 |

Manufacturing Process:

1) Orlistat, dibasic calcium phosphate anhydrous, and alginic acid were sifted through an ASTM #40 mesh sieve.

2) Step 1) materials were dry mixed for about 3 minutes.

3) Sodium alginate and Tween 80 were dissolved in water to form a binder solution

4) Step 2) materials were granulated using binder solution of step 3).

5) The wet granules were dried in a tray drier until loss on drying was below 1% w/w.

6) Colloidal silicon dioxide was sifted through an ASTM #40 mesh sieve, mixed with dried granules and blended for about 5 minutes.

7) The blend of 6) was filled into hard gelatin capsules.

### Example 11

### Powder Composition Comprising Orlistat

| Ingredient | Grams |
|---|---|
| Orlistat | 20 |
| Microcrystalline cellulose 112 (MCC 112, D(0.5) = 126 μm) | 300 |
| Isopropyl alcohol* | 25 |
| Methylene chloride* | 75 |

*Evaporates during processing.

Manufacturing Process:

1) Orlistat was combined with a mixture of isopropyl alcohol and methylene chloride and stirred to form a clear solution.

2) MCC 112 was loaded into a fluid bed coater and solution from step 1) was sprayed onto the MCC 112.

3) The material from step 2) was dried at an inlet temperature of 30 to 45° C. and bed temperature of 25 to 30° C. for about 1 hour, yielding a fine powder having the appearance of the original MCC 112.

4) Powder of step 3) was filled into hard gelatin capsules.

1. A pharmaceutical composition comprising an orlistat powder having an average particle size less than about 250 μm.

2. The pharmaceutical composition of claim 1, wherein an orlistat powder has an average particle size less than about 200 μm.

3. The pharmaceutical composition of claim 1, having a bulk density between about 0.2 and about 0.6 g/ml.

4. The pharmaceutical composition of claim 1, having a tapped density between about 0.3 and about 0.8 g/ml.

5. The pharmaceutical composition of claim 1, having an angle of repose not more than about 40.

6. A solid pharmaceutical formulation comprising the composition of claim 1, having an average particle size less than about 250 μm.

7. A solid pharmaceutical formulation comprising the composition of claim 1, having an average particle size greater than about 2 μm.

8. The solid pharmaceutical formulation of claim 6, having a unit weight within the limits of ±10% of an average weight of 20 units of the formulation.

9. The solid pharmaceutical formulation of claim 6, having a moisture content less than about 7% w/w.

10. A process for the preparation of a solid pharmaceutical formulation of claim 6, comprising:
a) mixing orlistat and at least one pharmaceutical excipient; and
b) compressing the mixture or filling the mixture into capsules.

11. A pharmaceutical formulation comprising orlistat, wherein the formulation has a delactone orlistat impurity concentration less than about 2% by weight of a label orlistat content.

12. A pharmaceutical formulation comprising orlistat, wherein the formulation has a concentration of total impurities less than about 4% by weight of a label orlistat content.
